(19) United States
(12) Patent Application Publication
(10) Pub. No.: US 2017/0084838 A1
(43) Pub. Date: Mar. 23, 2017

(54) ELECTRONIC DEVICE AND METHOD FOR MANUFACTURING THE SAME

(71) Applicant: InnoLux Corporation, Miao-Li County (TW)

(72) Inventors: Chi-Che TSAI, Miao-Li County (TW); Po-Ching LIN, Miao-Li County (TW); Po-Yun HSU, Miao-Li County (TW); Szu-Yu LAI, Miao-Li County (TW)

(21) Appl. No.: 14/857,929

(22) Filed: Sep. 18, 2015

(51) Int. Cl. H01L 51/00; H01L 51/52; H01L 51/56

(52) U.S. Cl. CPC H01L 51/003; H01L 51/56; H01L 51/5246

(57) ABSTRACT

An electronic device and a method for manufacturing the same are disclosed. The electronic device of the present disclosure comprises: a target unit comprising an electronic unit layer; and small molecule residues adhered on a side of the target unit, wherein the small molecule residues are at least one selected from the group consisting of

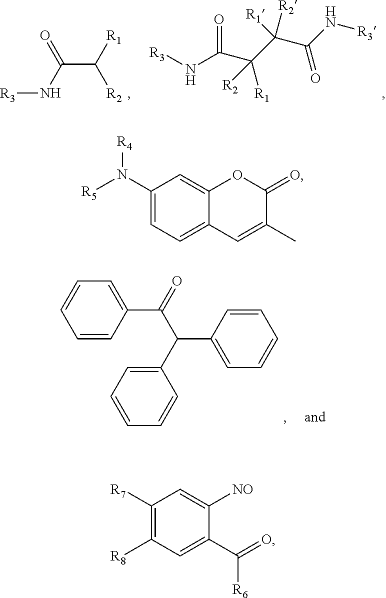

and R₁, R₁′, R₂, R₂′, R₃, R₃′, R₄, R₅, R₆, R₇, R₈ are defined in the specification.

### BACKGROUND

1. Field of the Invention

The present disclosure relates to an electronic device and a method for manufacturing the same and, more particularly, to a method for manufacturing an electronic device with a UV release layer and an electronic device manufactured by the method of the present disclosure.

2. Description of Related Art

As the demand for thin and light electronic devices, conventional glass substrate of the display panel is replaced by flexible substrate which is a thin glass having a thickness between 0.01 mm to 0.3 mm or a plastic substrate.

However, rigidity of the flexible substrate is not high enough for the current used process. Therefore, it is hard to form electronic units thereon through the current used process for manufacturing the electronic device.

In order to obtain the electronic device with the flexible substrate manufactured through the current used process, the flexible substrate is loaded on another carrier to increase the rigidity thereof, and then the flexible substrate is separated from the carrier after finishing electronic unit formation process.

On the other hand, the plastic substrate is also used for preparing a flexible organic light emitting diode (OLED) display device. In the process for manufacturing the OLED display device with the plastic substrate, a sealant layer is formed on a carrier via the conventional thermal release layer, and then transferred onto OLED units. After the sealant layer is cured, a thermal treatment is used to de-bond the carrier from the obtained OLED display device. However, the heat used for curing the sealant layer may cause the thermal release layer degraded, resulting in the OLED unit and/or the sealant layer uneven.

Therefore, it is desirable to provide a novel method for manufacturing an electronic device (including the OLED display device), wherein the carrier can be removed easily without damaging the obtained electronic device.

### SUMMARY

The object of the present disclosure is to provide a method for manufacturing an electronic device by using a UV release layer comprising a gas-releasing molecule. After UV irradiation, the gas-releasing molecule can be degraded to generate gas; and therefore, the carrier used in the manufacturing process can be easily removed by degrading the UV release layer. In addition, the present disclosure also provides an electronic device obtained by the method of the present disclosure.

To achieve the object, the method for manufacturing an electronic device comprises the following steps: providing a target unit and a carrier with a UV release layer formed thereon, wherein the carrier is adhered to the target unit, the UV release layer is disposed between the target unit and the carrier, the UV release layer comprises a polymer and a gas-releasing molecule, and the target unit comprises an electronic unit layer; and irradiating the UV release layer with UV light to degrade the gas-releasing molecule and separate the target unit from the carrier to obtain an electronic device. Herein, the gas-releasing molecule comprises at least one selected from the group consisting of

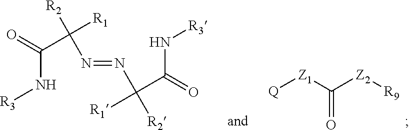

wherein each of R₁, R₁′, R₂, R₂′ and R₉ independently, is C₁₋₁₀ alkyl;
each of R₃ and R₃′ independently, is C₁₋₁₀ alkyl, C₂₋₁₀ alkenyl, or C₃₋₂₀ cycloalkyl;

### Z₁ is O;

### Z₂ is N or S; and

### Q is

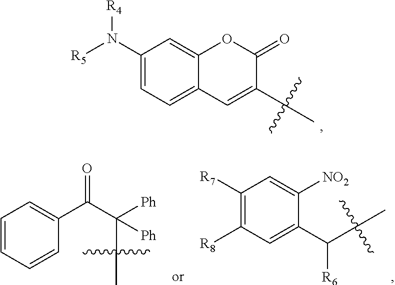

in which each of R₄, and
R₅ independently, is C₁₋₁₀ alkyl; R₆ is H or C₁₋₁₀ alkyl; and each of R₇ and R₈ independently, is C₁₋₁₀ alkoxy.

After the target unit is separated from the carrier, small molecule residues degraded from the UV release layer may remain on the target unit. Hence, after the method of the present disclosure, and electronic device can be obtained, which comprises: a target unit comprising an electronic unit layer; and small molecule residues adhered on a side of the target unit. Herein, the small molecule residues are at least one selected from the group consisting of

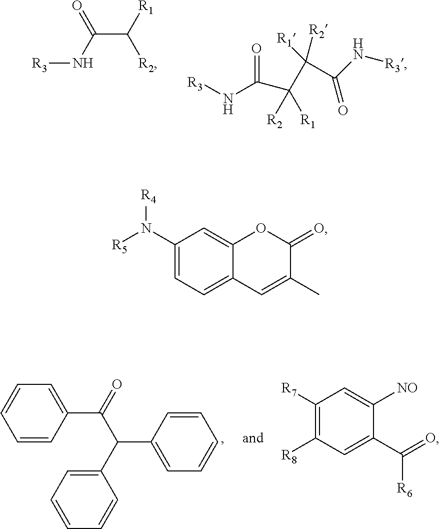

wherein each of R₁, R₁′, R₂, R₂′, R₄, and R₅ independently, is C₁₋₁₀ alkyl;
each of R₃ and R₃′ independently, is C₁₋₁₀ alkyl, C₂₋₁₀ alkenyl, or C₃₋₂₀ cycloalkyl;
R₆ is H or C₁₋₁₀ alkyl; and
each of R₇ and R₈ independently, is C₁₋₁₀ alkoxy.

In addition, in the method of the present disclosure, preferably, the gas-releasing molecule comprises at least one selected from the group consisting of

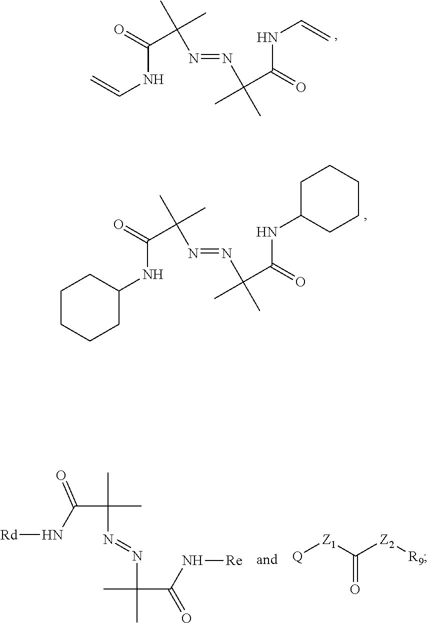

wherein each of Rd and Re independently, is C₂₋₆ alkyl;
R₉ is C₁₋₅ alkyl;

### Z₁ is O;

### Z₂ is N or S; and

### Q is

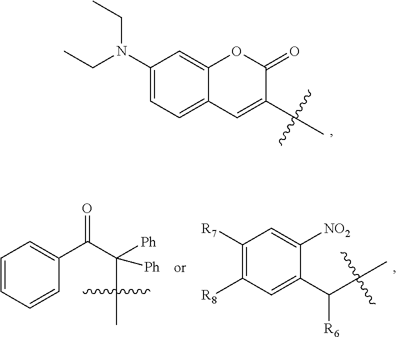

in which R₆ is H or methyl; and each of R₇ and R₈ independently, is methoxy or ethoxy.

When the gas-releasing molecule comprises the aforementioned preferable molecules, the small molecule residues remained on the electronic device after UV irradiation preferably are at least one selected from the group consisting of

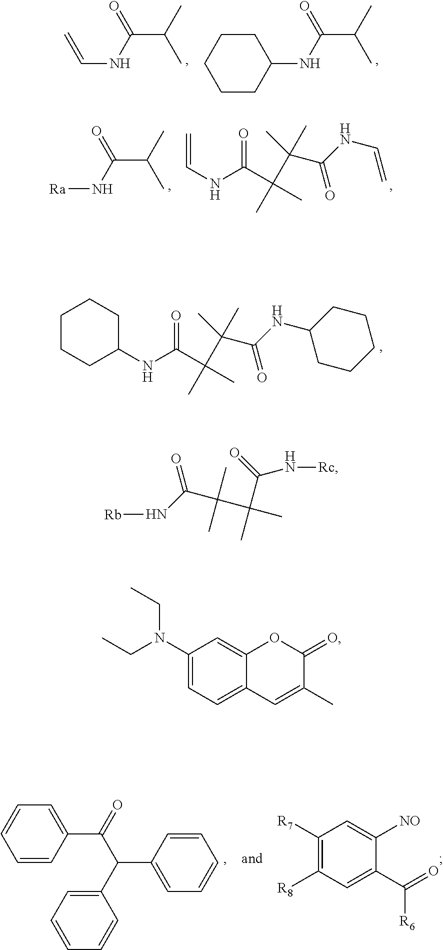

and
wherein each of Ra, Rb and Rc independently, is C₂₋₆ alkyl;
R₆ is H or methyl; and
each of R₇ and R₈ independently, is methoxy or ethoxy.

In the present disclosure, alkyl, alkenyl, cycloalkyl, alkoxy, phenyl present in the small molecule residues and the gas-releasing molecule include both substituted and unsubstituted moieties, unless specified otherwise. Possible substituents on alkyl, alkenyl, cycloalkyl and alkoxy include, but are not limited to, alkyl, halogen, alkoxy, heterocyclic group or aryl; but alkyl cannot be substituted with alkyl.

In the present disclosure, the term “halogen” includes F, Cl, Br and I; and preferably is F, Cl or Br. The term “alkyl” refers to linear and branched alkyl; preferably, includes linear or branched C₁₋₁₀ alkyl; and more preferably, includes linear or branched C₁₋₆ alkyl. Specific examples of alkyl include, but are not limited to, methyl, ethyl, n-propyl, iso-propyl, n-butyl, sec-butyl, iso-butyl, tert-butyl, pentyl, neo-pentyl or hexyl. The term “alkenyl” refers to a linear or branched hydrocarbon moiety that contains at least one double bond; preferably, includes a linear or branched hydrocarbon C₂₋₁₀ moiety containing at least one double bond; and more preferably, includes a linear or branched hydrocarbon C₂₋₆ moiety containing at least one double bond. Specific examples of alkenyl include, but are not limited to, ethenyl, propenyl, allyl, or 1,4-butadienyl. The term “alkoxy” refers to a moiety that the alkyl defined in the present disclosure coupled with an oxygen atom; preferably, includes linear or branched C₁₋₁₀ alkoxy; and more preferably, includes linear or branched C₁₋₆ alkoxy. Specific examples of alkoxy include, but are not limited to, methoxy, ethoxy, n-propoxy, iso-propoxy, n-butoxy, iso-butoxy, sec-butoxy, tert-butoxy, pentyloxy, neo-pentyloxy or hexyloxy. The term “cycloalkyl” refers to a monovalent saturated hydrocarbon ring system having 3 to 20 carbon atoms; and preferably having 3 to 12 carbon atoms. Specific examples of cycloalkyl include, but are not limited to, cyclopropyl, cyclobutyl, cyclopentyl, cyclohexyl, cycloheptyl, and cyclooctyl. The term “heterocyclic group” refers to a 5-8 membered monocyclic, 8-12 membered bicyclic or 11-14 membered tricyclic heteroaryl or heterocycloalkyl having at least one heteroatom which is selected from the group consisting of O, S and N. Specific examples of heterocyclic group include, but are not limited to, pyridyl, pyrimidinyl, furyl, thiazolyl, imidazolyl or thienyl. The term “aryl” refers to a monovalent 6-carbon monocyclic, 10-carbon bicyclic, or 14-carbon tricyclic aromatic ring system. Specific examples of aryl include, but are not limited to, phenyl, naphthyl, pyrenyl, anthracenyl or phenanthryl; and preferably, the aryl is phenyl.

In the method of the present disclosure, the conventional thermal release layer is substituted with a UV release layer, which does not degrade during the curing process of the sealant layer; therefore, the aforementioned problem can be solved. Other objects, advantages, and novel features of the disclosure will become more apparent from the following detailed description when taken in conjunction with the accompanying drawings.

### BRIEF DESCRIPTION OF THE DRAWINGS

FIGS. 1A to 1D are cross-sectional view showing a process for manufacturing an OLED display device according to Embodiment 1 of the present disclosure.

FIGS. 2A to 2D are cross-sectional view showing a process for manufacturing an electronic device according to Embodiment 2 of the present disclosure.

FIG. 3 is a cross-sectional view of a testing sheet used in Texting examples 1 and 2 of the present disclosure.

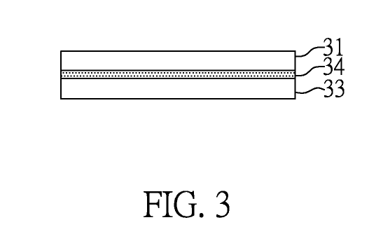

### DETAILED DESCRIPTION OF THE PREFERRED EMBODIMENT

The present disclosure has been described in an illustrative manner, and it is to be understood that the terminology used is intended to be in the nature of description rather than of limitation. Many modifications and variations of the present disclosure are possible in light of the above teachings. Therefore, it is to be understood that within the scope of the appended claims, the disclosure may be practiced otherwise than as specifically described.

In the following embodiments and other embodiments of the present disclosure, alkyl, alkenyl, cycloalkyl, alkoxy, phenyl present in the small molecule residues and the gas-releasing molecule include both substituted and unsubstituted moieties, unless specified otherwise. Possible substituents on alkyl, alkenyl, cycloalkyl and alkoxy include, but are not limited to, alkyl, halogen, alkoxy, heterocyclic group or aryl; but alkyl cannot be substituted with alkyl.

In addition, in the following embodiments and other embodiments of the present disclosure, the term “halogen” includes F, Cl, Br and I; and preferably is F, Cl or Br. The term “alkyl” refers to linear and branched alkyl; preferably, includes linear or branched C₁₋₁₀ alkyl; and more preferably, includes linear or branched C₁₋₆ alkyl. Specific examples of alkyl include, but are not limited to, methyl, ethyl, n-propyl, iso-propyl, n-butyl, sec-butyl, iso-butyl, tert-butyl, pentyl, neo-pentyl or hexyl. The term “alkenyl” refers to a linear or branched hydrocarbon moiety that contains at least one double bond; preferably, includes a linear or branched hydrocarbon C₂₋₁₀ moiety containing at least one double bond; and more preferably, includes a linear or branched hydrocarbon C₂₋₆ moiety containing at least one double bond. Specific examples of alkenyl include, but are not limited to, ethenyl, propenyl, allyl, or 1,4-butadienyl. The term “alkoxy” refers to a moiety that the alkyl defined in the present disclosure coupled with an oxygen atom; preferably, includes linear or branched C₁₋₁₀ alkoxy; and more preferably, includes linear or branched C₁₋₆ alkoxy. Specific examples of alkoxy include, but are not limited to, methoxy, ethoxy, n-propoxy, iso-propoxy, n-butoxy, iso-butoxy, sec-butoxy, tert-butoxy, pentyloxy, neo-pentyloxy or hexyloxy. The term “cycloalkyl” refers to a monovalent saturated hydrocarbon ring system having 3 to 20 carbon atoms; and preferably having 3 to 12 carbon atoms. Specific examples of cycloalkyl include, but are not limited to, cyclopropyl, cyclobutyl, cyclopentyl, cyclohexyl, cycloheptyl, and cyclooctyl. The term “heterocyclic group” refers to a 5-8 membered monocyclic, 8-12 membered bicyclic or 11-14 membered tricyclic heteroaryl or heterocycloalkyl having at least one heteroatom which is selected from the group consisting of O, S and N. Specific examples of heterocyclic group include, but are not limited to, pyridyl, pyrimidinyl, furyl, thiazolyl, imidazolyl or thienyl. The term “aryl” refers to a monovalent 6-carbon monocyclic, 10-carbon bicyclic, or 14-carbon tricyclic aromatic ring system. Specific examples of aryl include, but are not limited to, phenyl, naphthyl, pyrenyl, anthracenyl or phenanthryl; and preferably, the aryl is phenyl.

### Embodiment 1

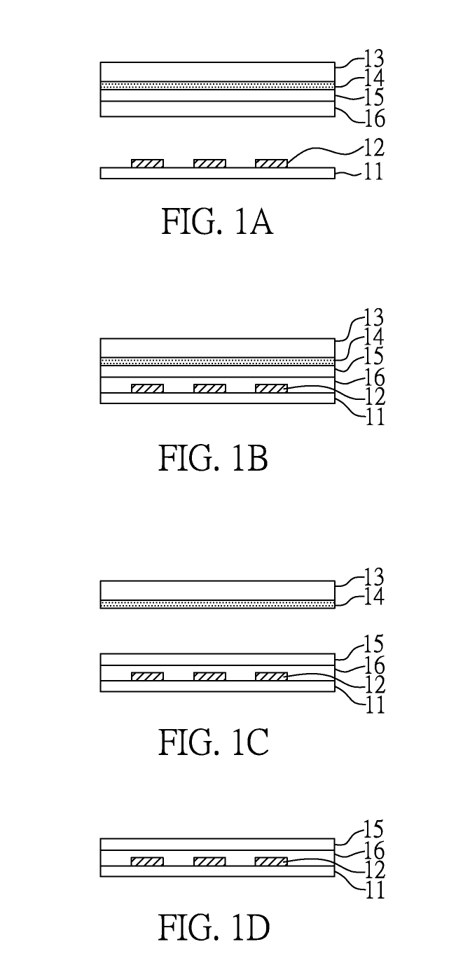

FIGS. 1A to 1D are cross-sectional view showing a process for manufacturing an OLED display device according to the present embodiment.

As shown in FIG. 1A, a target unit comprising a substrate 11 with an OLED unit 12 (as an electronic unit layer) formed thereon is provided; and a carrier 13 with a UV release layer 14 formed thereon is also provided. Herein, the UV release layer 14 can be formed on the carrier 13 by a coating process such as a spin coating process, a dip coating process, a blade coating process, a printing process and so on; or the UV release layer 14 can be formed in a tap manner in advance, and then the UV release layer 14 is adhered onto the carrier 13 through a rolling process.

In the present embodiment, the material of the carrier 13 is not particularly limited; as long as the carrier can be applied on the current used machine in the art, for example, a glass carrier, a quartz carrier, or a plastic carrier. Preferably, the carrier 13 has a light transmittance larger than 30%. More preferably, the light transmittance of the carrier is in a range from 80% to 99%. Most preferably, the light transmittance thereof is in a range from 90% to 99%. Specifically, the carrier 13 preferably has a light transmittance larger than 30% under UV irradiation (which has a wavelength of 200-420 nm). More preferably the light transmittance of the carrier 13 is in a range from 80% to 99% under UV irradiation. Most preferably, the light transmittance thereof is in a range from 90% to 99% under UV irradiation.

In the present embodiment, a material of the UV release layer 14 comprises a polymer and a gas-releasing molecule. In addition, a photo initiator is also contained in the material of the UV release layer 14.

Herein, the polymer contained in the UV release layer 14 is preferably has the following properties. The first one is that the polymer can provide enough adhesion for the carrier 13. The second one is that the adhesion thereof is decreased after the UV irradiation; in particular, after the UV irradiation, the polymer is cross-linked, the loss factor (adhesive behavior) tan δ decreases and thus the adhesion thereof is decreased. Wherein the loss factor tan δ=G″/G′, G′ is storage modulus (elastic behavior), and G″ is loss modulus (viscous behavior). In the present embodiment, specific examples of the polymer include, but are not limited to acrylic polymer.

In addition, the gas-releasing molecule contained in the UV release layer 14 of the present embodiment may comprise: at least one selected from the group consisting of

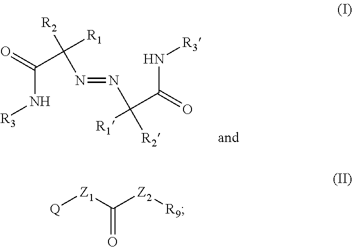

wherein each of R₁, R₁′, R₂, R₂′ and R₉ independently, is C₁₋₁₀ alkyl;
each of R₃ and R₃′ independently, is C₁₋₁₀ alkyl, C₂₋₁₀ alkenyl, or C₃₋₂₀ cycloalkyl;

### Z₁ is O;

### Z₂ is N or S; and

### Q is

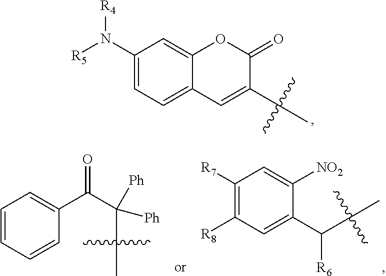

in which each of R₄, and
R₅ independently, is C₁₋₁₀ alkyl; R₆ is H or C₁₋₁₀ alkyl; and each of R₇ and R₈ independently, is C₁₋₁₀ alkoxy.

Specific examples of the gas-releasing molecule include, but are not limited to

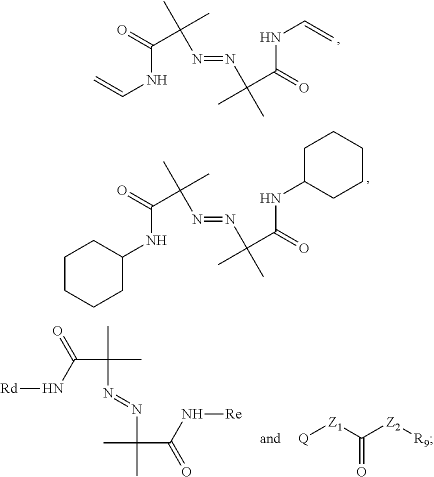

wherein each of Rd and Re independently, is C₂₋₆ alkyl;
R₉ is C₁₋₅ alkyl;

### Z₁ is O;

### Z₂ is N or S; and

### Q is

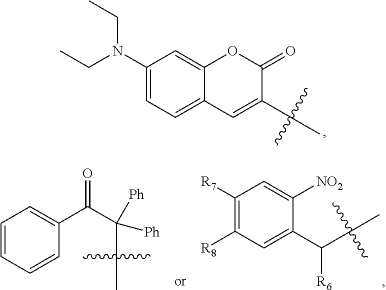

in which R₆ is H or methyl; and each of R₇ and R₈ independently, is methoxy or ethoxy.

The gas-releasing molecules represented by the formulas (I) and (II) can respectively release N₂ and CO₂ after UV irradiation. Herein, the gas-releasing molecules represented by the formulas (I) and (II) can be used alone or in combination. In addition, the wavelength of the UV light used in the UV irradiation is not particularly limited, and depends upon the substituents present in the formulas (I) and/or (II).

Herein, the thickness of the UV release layer 14 is 10˜100 μm. If the thickness of the UV release layer 14 is too thin, the UV release layer 14 cannot provide enough adhesion for adhering the target unit and the carrier 13. If the thickness thereof is too thick, the polymer contained in the UV release layer 14 may be remain on the obtained electronic device.

In the present embodiment, as shown in FIG. 1A, a sealant layer 16 is further disposed on the UV release layer 14, and the UV release layer 14 is disposed between the carrier 13 and the sealant layer 16. Herein, a sealant layer 16 is a face sealant used in the OLED display device. In addition, in the present embodiment, a barrier layer 15 is also formed on the UV release layer 14, and the barrier layer 15 is disposed between the UV release layer 14 and the sealant layer 16.

In the present embodiment, the substrate 11 is a plastic substrate such as a poly(ethylene terephthalate) (PET) substrate, a polyethylene naphthalate (PEN) substrate and a cyclic olefin copolymer (COP) substrate, but the present disclosure is not limited thereto. In other embodiment, the substrate 11 is a thin glass substrate. Herein, the OLED unit 12 shown in FIGS. 1A to 1D is simplified into one single layer; however, even though not shown in the figure, the OLED unit 12 of the present embodiment may comprise: TFT units; electrode layers containing anode and cathode; conducting wires containing scan lines, data lines, and power lines; a pixel define layer; capping layers; encapsulation layers; OLED layers containing a light emitting layer, an electron transporting layer, an electron injection layer, a hole transporting layer, a hole injection layer, and/or other layers capable of facilitating the combination of holes and electrons.

After the carrier 13 and the target unit comprising the substrate 11 with the OLED unit 12 formed thereon are provided, the carrier 13 is adhered to the target unit, the UV release layer 14 is disposed between the target unit and the carrier 13, as shown in FIG. 1B. More specifically, a side of the OLED unit 12 is adhered to the sealant layer 16 on the UV release layer 14, and the UV release layer 14 locates between the OLED unit 12 and the carrier 13. Then, after the sealant layer 16 is cured by heating, UV light is applied onto the carrier 13 and penetrates through the carrier 13 to achieve the UV release layer 14 to degrade the gas-releasing molecule in the UV release layer 14, and thus the target unit comprising the substrate 11 and the OLED unit 12 can be separated from the carrier 13 (as shown in FIG. 1C) to obtain an OLED display device (as shown in FIG. 1D) in which the sealant layer 16 as well as the barrier layer 15 are transferred onto the OLED unit 12.

Herein, the UV irradiation is provided from the side of the carrier 13. More specifically, the radiation is provided onto the carrier 13 without the UV release layer 14 formed thereon, so that the light can penetrate through the carrier 13 to achieve the UV release layer 14 to perform the photo-reaction (the photo-cleavage, or the photo-degradation). Herein, the wavelength, the light intensity, and the UV irradiation time are not particularly limited, and can be selected according to the gas-releasing molecule, as long as the separation between the target unit and the carrier 13 can be achieved.

After the UV release layer 14 is degraded and the carrier 13 is removed, small molecule residues degraded from the UV release layer 14 may remain on the target unit (especially on the barrier layer 15), which are at least one selected from the group consisting of

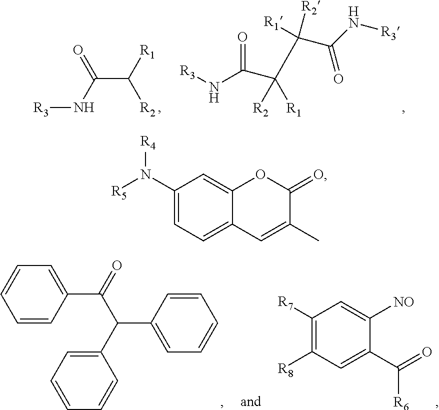

wherein each of R₁, R₁′, R₂, R₂′, R₄, and R₅ independently, is C₁₋₁₀ alkyl;
each of R₃ and R3′ independently, is C₁₋₁₀ alkyl, C₂₋₁₀ alkenyl, or C₃₋₂₀ cycloalkyl;
R₆ is H or C₁₋₁₀ alkyl; and
each of R₇ and R₈ independently, is C₁₋₁₀ alkoxy.

Herein, specific examples of the small molecule residues include, but are not limited to

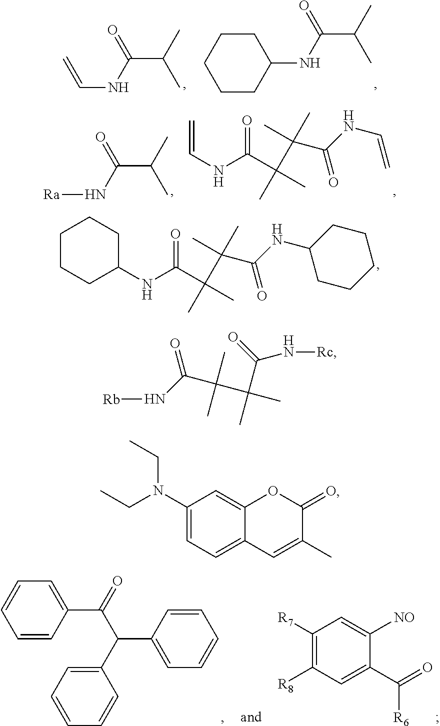

and
wherein each of Ra, Rb and Rc independently, is C₂₋₆ alkyl;
R₆ is H or methyl; and
each of R₇ and R₈ independently, is methoxy or ethoxy.

After the aforementioned process, as shown in FIG. 1D, an OLED display device of the present embodiment is provided, which comprises: a target unit comprising the substrate 11 with the OLED unit 12 (as an electronic unit layer) formed thereon; and small molecule residues adhered on a side of the target unit. More specifically, in the present embodiment, the small molecule residues are adhered on a side of the OLED unit 12, and particularly on a surface of the barrier layer 15.

Herein, it should be noted that, the UV release layer 14 is almost separated from the target unit (especially, the barrier layer 15 on the OLED unit 12) after the UV irradiation, and only small molecule residues are maintained on the surface of the target unit.

In the present embodiment, as shown in FIGS. 1A to 1D, for the flexible OLED display device, the used substrate 11 is a plastic substrate and the sealant layer 16 (which is usually a face sealant) is transferred from the carrier 13 onto the OLED unit 12. In the conventional process, the sealant layer 16 is adhered on the carrier 13 via the conventional thermal release layer in advance, the carrier 13 is laminated onto the substrate 11 by facing the sealant layer 16 to the OLED unit 12, and then the sealant layer 16 is cured by heating and pressing. However, the heat for curing the sealant layer 16 may cause the conventional thermal release layer gradually degraded. The degraded thermal release layer may loss its adhesion property; and the gas released from the degraded thermal release layer may compress OLED unit 12 and/or the sealant layer 16, causing the OLED unit 12 and/or the sealant layer 16 uneven.

In the present embodiment, as shown in FIGS. 1A to 1D, the UV release layer 14, which is not degraded by heat, is used to replace the conventional thermal release layer. Therefore, during the curing process of the sealant layer 16, the UV release layer 14 does not degrade and the adhesion thereof can be maintained. When the curing process of the sealant layer 16 is completed, the UV light is applied onto the UV release layer 14. During the UV irradiation, the polymer contained in the UV release layer 14 is gradually cross-linked, resulting in the adhesion thereof decreased. In addition, the gas-releasing molecule contained in the UV release layer 14 is gradually degraded due to the UV irradiation, and the gas degraded from the gas-releasing molecule can expand and open up the adhesion interface between the UV release layer 14 and the OLED unit 12 (in the present embodiment, the barrier layer 15). Therefore, the debonding of the carrier 13 can be accomplished. Since the UV release layer 14 does not degrade and there is no gas generated during the curing process of the sealant layer 16, the aforementioned problem resulting from using the conventional thermal release layer can be eliminated.

### Embodiment 2

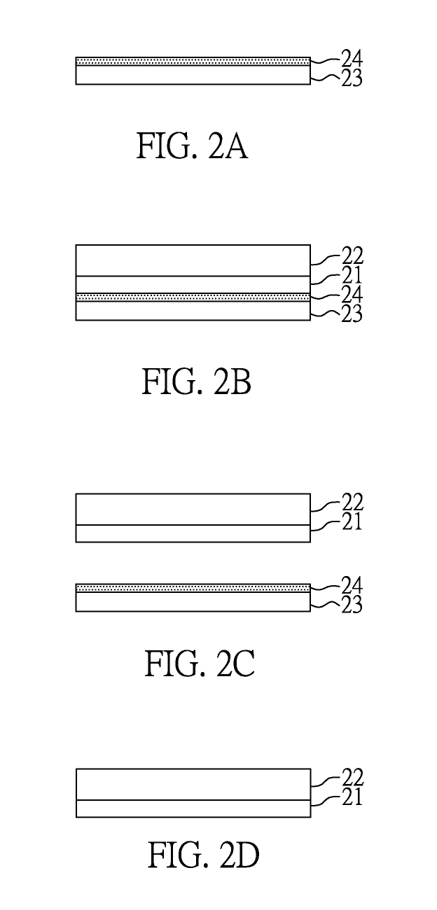

FIGS. 2A to 2D are cross-sectional view showing a process for manufacturing an electronic device according to the present embodiment.

As shown in FIG. 2A, a carrier 23 with a UV release layer 24 formed thereon is provided. Next, as shown in FIG. 2B, a substrate 21 and an electronic unit layer 22 are sequentially laminated on the UV release layer 24 to form a target unit. Herein, the carrier 23 is adhered to the target unit, and the UV release layer 24 is disposed between the carrier 23 and the target unit comprising the substrate 21 and the electronic unit layer 22. More specifically, a side of the substrate 21 is adhered to the UV release layer 24, and the UV release layer 24 locates between the substrate 21 and the carrier 23.

In the present embodiment, the substrate 21 is a flexible substrate, such as a plastic substrate, a thin glass substrate with a thickness of 0.3 mm or less, a metal substrate, and a stainless steel substrate. The flexible substrate does not have enough rigidity, so the carrier 23 can be used as a support for the sequential process for forming the electronic unit layer 22.

Herein, the carrier 23 and the UV release layer 24 are similar to those illustrated in Embodiment 1, and therefore the descriptions thereof are not repeated.

Then, as shown in FIG. 2C, UV light is applied onto the carrier 23 and penetrates through the carrier 23 to achieve the UV release layer 24 to degrade the gas-releasing molecule in the UV release layer 24, and thus the target unit comprising the substrate 21 and the electronic unit layer 22 can be separated from the carrier 23, as shown in FIG. 2D.

After the UV release layer 24 is degraded and the carrier 23 is removed, small molecule residues degraded from the UV release layer 24 may remain on the target unit (especially on a surface of the substrate 21). Herein, the small molecule residues on the substrate 21 are similar to those illustrated in Embodiment 1, and therefore the descriptions thereof are not repeated.

After the aforementioned process, as shown in FIG. 2D, an electronic device of the present embodiment is provided, which comprises: a target unit comprising the substrate 21 and the electronic unit layer 22; and small molecule residues adhered on a side of the target unit. More specifically, in the present embodiment, the small molecule residues are adhered on a side of the substrate 21, but not on a side of the electronic unit layer 22.

In the present embodiment, as shown in FIGS. 2A to 2D, for the flexible electronic device, the used flexible substrate 21 does not have enough rigidity, and therefore, a carrier 23 has to be used to support the flexible substrate for the sequential process. If the flexible substrate is adhered onto the carrier via the conventional thermal release layer, the sequential high temperature process may cause the adhesion of the thermal release layer deteriorated, and thus the manufacturing process may be failed.

In the present embodiment, as shown in FIGS. 2A to 2D, the UV release layer 24, which is not degraded by heat, is used to replace the conventional thermal release layer. Even though the heat is generated during the sequential high temperature process, the UV release layer 24 still can keep its adhesion property, and therefore the aforementioned problem caused by the using of the thermal release layer can be solved.

### Test Example 1

In the present test example, the used testing sheet comprises: a substrate 31; a carrier 33; and a UV release layer 34 sandwiched between a carrier 33 and a substrate 31. Herein, the substrate 31 and the carrier 33 are glass substrates; and the UV release layer 34 comprises acrylic acid polymer and methacrylic polymer and a gas-releasing molecule represented by the following formula (I-1):

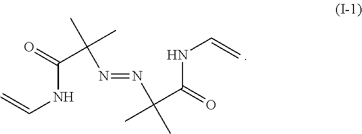 [SMILES: C=CNC(=O)C(C)(C)/N=N/C(C)(C)C(=O)NC=C]

After UV light having a wavelength of 375 nm is applied onto the UV release layer 34, a photo-degradation process is progressed in the UV release layer 34 to degrade the compound of the formula (I-1); and

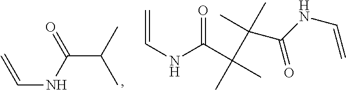

and N₂ are generated. Bubbles can be observed in the testing sheet (the photo not shown), which are caused by the generated N₂.

### Test Example 2

The testing sheet used in the present test example is similar to that used in Test example 1, except that the gas-releasing molecule used in the present Test example is represented by the following formula (II-1):

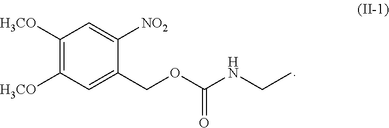 [SMILES: CCNC(=O)OCc1cc(OC)c(OC)cc1[N+](=O)[O-]]

After UV light having a wavelength of 365 nm is applied onto the UV release layer 34,

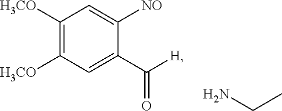

and CO₂ degraded from the compound of formula (II-1) are generated. Bubbles can be observed in the testing sheet (the photo not shown), which are caused by the generated CO₂.

The results shown in Test examples 1 and 2 indicate that the gas can generate due to the photo-degradation process of the gas-releasing molecule, and the generated gas can expand and open up the adhesion interface between the UV release layer and the substrate.

In the present disclosure, the electronic device obtained in the present embodiment can also be applied to various apparatus, such as cell phones, notebooks, video cameras, cameras, music players, navigation devices, and televisions.

Although the present disclosure has been explained in relation to its preferred embodiment, it is to be understood that many other possible modifications and variations can be made without departing from the spirit and scope of the disclosure as hereinafter claimed.

What is claimed is:

1. An electronic device, comprising:
a target unit comprising an electronic unit layer; and
small molecule residues adhered on a side of the target unit,
wherein the small molecule residues are at least one selected from the group consisting of 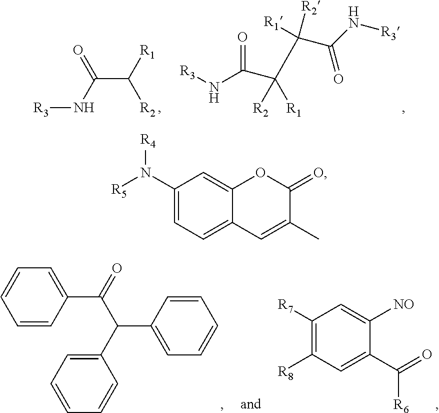
wherein each of R₁, R₁′, R₂, R₂′, R₄, and R₅ independently, is C₁₋₁₀ alkyl;
each of R₃ and R₃′ independently, is C₁₋₁₀ alkyl, C₂₋₁₀ alkenyl, or C₃₋₂₀ cycloalkyl;
R₆ is H or C₁₋₁₀ alkyl; and
each of R₇ and R₈ independently, is C₁₋₁₀ alkoxy.

2. The electronic device of claim 1, wherein the target unit comprises a substrate with the electronic unit layer formed thereon, wherein the small molecule residues are adhered on a side of the substrate.

3. The electronic device of claim 1, wherein the target unit comprises a substrate with an OLED unit formed thereon, and the small molecule residues are adhered on a side of the OLED unit.

4. The electronic device of claim 1, wherein the small molecule residues are at least one selected from the group consisting of 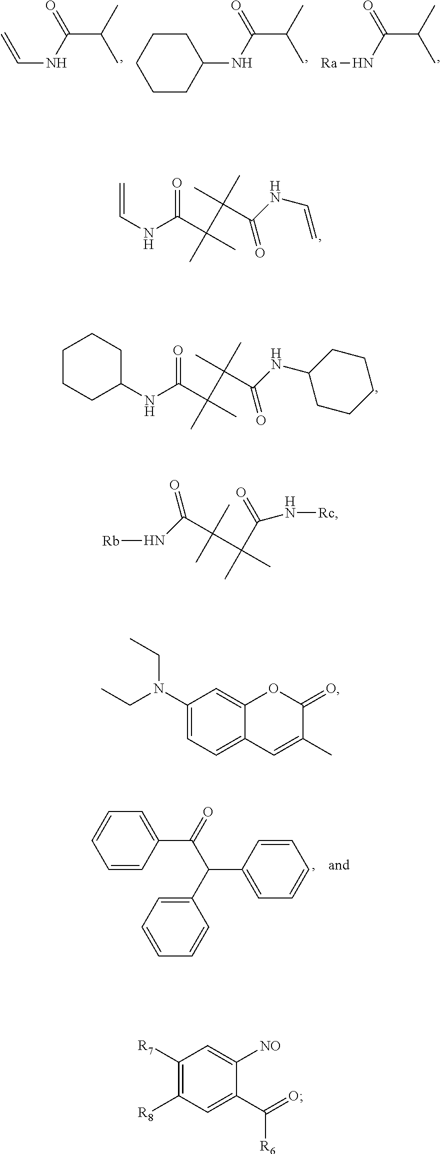
and
wherein each of Ra, Rb and Rc independently, is C₂₋₆ alkyl;
R₆ is H or methyl; and
each of R₇ and R₈ independently, is methoxy or ethoxy.

5. A method for manufacturing an electronic device, comprising the following steps:
providing a target unit and a carrier with a UV release layer formed thereon, wherein the carrier is adhered to the target unit, the UV release layer is disposed between the target unit and the carrier, the UV release layer comprises a polymer and a gas-releasing molecule, and the target unit comprises an electronic unit layer; and
irradiating the UV release layer with UV light to degrade the gas-releasing molecule and separate the target unit from the carrier to obtain an electronic device,
wherein the gas-releasing molecule comprises at least one selected from the group consisting of 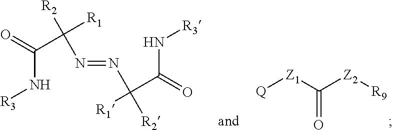
wherein each of R₁, R₁′, R₂, R₂′ and R₉ independently, is C₁₋₁₀ alkyl;
each of R₃ and R₃′ independently, is C₁₋₁₀ alkyl, C₂₋₁₀ alkenyl, or C₃₋₂₀ cycloalkyl;
Z₁ is O;
Z₂ is N or S; and
Q is 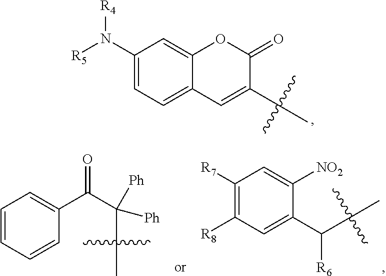
in which each of R₄, and R₅ independently, is C₁₋₁₀ alkyl; R₆ is H or C₁₋₁₀ alkyl; and each of R₇ and R₈ independently, is C₁₋₁₀ alkoxy.

6. The method of claim 5, wherein the gas-releasing molecule comprises at least one selected from the group consisting of 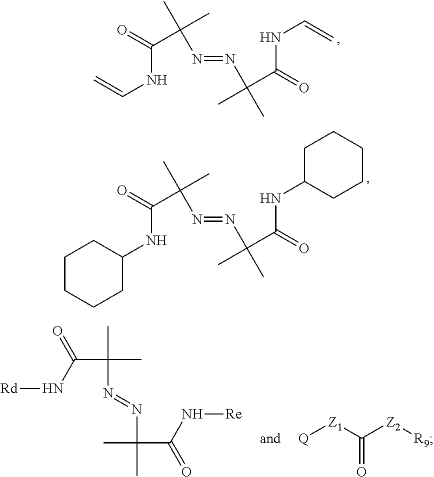
wherein each of Rd and Re independently, is C₂₋₆ alkyl;
R₉ is C₁₋₅ alkyl;
Z₁ is O;
Z₂ is N or S; and
Q is 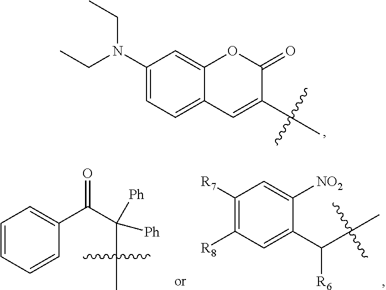
in which R₆ is H or methyl; and each of R₇ and R₈ independently, is methoxy or ethoxy.

7. The method of claim 5, wherein the target unit comprises a substrate with an electronic unit layer formed thereon, a side of the substrate is adhered to the UV release layer, and the UV release layer locates between the substrate and the carrier.

8. The method of claim 5, wherein the target unit comprises a substrate with an OLED unit as the electronic unit layer formed thereon, a side of the OLED unit is adhered to the UV release layer, and the UV release layer locates between the OLED unit and the carrier.

9. The method of claim 8, wherein in the step of providing the target unit and the carrier, a sealant layer is further disposed on the UV release layer, and the UV release layer is disposed between the carrier and the sealant layer; and in the step of irradiating the UV release layer with the UV light, the target unit is separated from the carrier and the sealant layer is transferred onto the OLED unit.

10. The method of claim 9, wherein in the step of providing the target unit and the carrier, a barrier layer is further disposed between the sealant layer and the UV release layer; and in the step of irradiating the UV release layer with the UV light, the barrier layer is transferred onto the OLED unit.
